(19) United States
(12) Patent Application Publication
(10) Pub. No.: US 2014/0220085 A1
(43) Pub. Date: Aug. 7, 2014

(54) METHOD FOR TISSUE ENGINEERING

(71) Applicant: Industrial Technology Research Institute, Chutung (TW)

(72) Inventors: Pei-Yi TSAI, Hsinchu City (TW); Yi-Hung WEN, Hsinchu City (TW); Zhi-Jie HUANG, Zhunan Township (TW); Pei-Shan LI, Taipei City (TW); Hsin-Hsin SHEN, Zhudong Township (TW); Yi-Hung LIN, Jhubei City (TW); Chih-Hung CHEN, Tainan City (TW)

(73) Assignee: Industrial Technology Research Institute, Chutung (TW)

(21) Appl. No.: 14/251,472

(22) Filed: Apr. 11, 2014

Related U.S. Application Data
(62) Division of application No. 13/605,361, filed on Sep. 6, 2012.

(30) Foreign Application Priority Data
Sep. 7, 2011 (TW) 100132196

(51) Int. Cl. A61L 27/54; A61L 27/10; C12N 5/00; A61L 27/12

(52) U.S. Cl. CPC A61L 27/54; A61L 27/12; A61L 27/10; C12N 5/0068
USPC 424/400; 514/8.8; 424/93.72; 514/1.1; 514/44 R; 424/520; 424/93.7; 424/93.71; 435/402; 435/396; 435/174; 435/178

(57) ABSTRACT

In an embodiment of the disclosure, a biomedical material is provided. The biomedical material includes a biocompatible material having a surface and a carrier distributed over the surface of the biocompatible material, wherein both of the biocompatible material and the carrier have no charges, one of them has charges or both of them have charges with different electricity. The biomedical material is utilized for dentistry, orthopedics, wound healing or medical beauty and applied in the repair and regeneration of various soft and hard tissues.

### CROSS REFERENCE TO RELATED APPLICATIONS

This application is a divisional of U.S. application Ser. No. 13/605,361, filed on Sep. 6, 2012, which claims priority of Taiwan Patent Application No. 100132196, filed on Sep. 7, 2011, the entirety of which is incorporated by reference herein.

### BACKGROUND

1. Technical Field

Technical field relates to a biomedical material for tissue engineering.

2. Description of the Related Art

The material for dental (or bone) defect repair used to be Cotton-Gauze-based materials (the first generation products) and biocompatible materials without active ingredients (the second generation products), such as β-TCP, hydroxyapatite, bioactive glass or collagen. And recently, some dental defect repairing products manufacturers (e.g., Nobel Biocare, Straumann, Biomet 3i, Zimmer Dental and Dentsply Friadent) have developed the third generation products which directly blended biocompatible materials and bioactive ingredients therefore conduced functions of anti-bacteria, anti-inflammatory and tissue regeneration. However, the above-mentioned products still have the problems of fill reflux and poor dental bone regeneration due to rapid denaturation or insufficient immobilization of bioactive substances. Therefore, how to effectively encapsulate bioactive substances and how to create effective osteoblast migration and binding of encapsulating materials are the issues expected to be resolved for current clinical treatment. Currently, although the active treatment technologies using biomedical carriers encapsulating growth factors (GF), for example, “Medtronic infuse” (e.g., the bovine collagen carrier type I comprising collagen sponge and collagen particles adsorbing rhBMP-2 proteins) have been developed, the encapsulation efficiency remains unsatisfactory. The actual amount of BMP-2 (which is very expensive: 300 USD/10 μg) adsorbed by the collagen sponge cannot be ascertained in clinical treatments. Therefore, the actual usage of BMP-2 generally exceeding the theoretically required therapeutic dosage amount, wherein the BMP-2 adsorbed by the collagen sponge will be easily released in a great quantity in a short time after entering into the body. Additionally, the shelf life of BMP-2 is short, so the storage can also be a problem. Also, growth factor (GF) is one kind of protein, which is easily denatured and degraded under acid, alkali and organic solvents and rapidly wash out in clinical application. Accordingly, many products have high concentration levels of GF to maintain effectiveness, and resulting in a lot of unexpected side effects.

### SUMMARY

An embodiment of the disclosure provides a biomedical material, comprising: a biocompatible material having a surface; and a carrier distributed over the surface of the biocompatible material, wherein both of the biocompatible material and the carrier have no charges, one of them has charges or both of them have charges with different electricity.

An embodiment of the disclosure provides a method for tissue engineering, comprising: providing the disclosed biomedical material; and applying the biomedical material to a targeted tissue for regeneration or healing or a medium for cell growth or cell adhesion.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawing, wherein:

FIG. 1 is a biomedical material in accordance with an embodiment of the disclosure;

FIG. 2 is a biomedical material in accordance with an embodiment of the disclosure;

FIG. 3 is a biomedical material in accordance with an embodiment of the disclosure;

FIG. 4 is a biomedical material capsule in accordance with an embodiment of the disclosure;

FIG. 5 is an effect of a nano carrier (phosphatidylcholine (PC)/cholesterol) encapsulating a bioactive substance (BMP-2) on the activity of an alkaline phosphatase (ALP/BCA) in accordance with an embodiment of the disclosure;

FIG. 6 is an effect of a nano carrier (phosphatidylcholine (PC)/vitamin) encapsulating a bioactive substance (BMP-2) on the activity of an alkaline phosphatase (ALP) in accordance with an embodiment of the disclosure;

FIG. 7 is an effect of a nano carrier (phosphatidylcholine (PC)/vitamin A) encapsulating a bioactive substance (BMP-2) on the activity of an alkaline phosphatase (ALP) in accordance with an embodiment of the disclosure;

FIG. 8 is alterations of contents of TGF-β1 of platelets rich plasma (PRP) with time in accordance with an embodiment of the disclosure;

FIG. 9 is alterations of contents of PDGF-AB of platelets rich plasma (PRP) with time in accordance with an embodiment of the disclosure;

FIG. 10 is a controlled-release curve of a biomedical material (agglomer) in accordance with an embodiment of the disclosure;

FIG. 11 is an effect of a biomedical material (agglomer) on the activity of an alkaline phosphatase (ALP) in accordance with an embodiment of the disclosure;

FIGS. 12A-12E are effects of a biomedical material (agglomer) and other control groups on bone repair in accordance with an embodiment of the disclosure; and

FIG. 13 is effects of a biomedical material (agglomer) and other control groups on increased bone volume in accordance with an embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

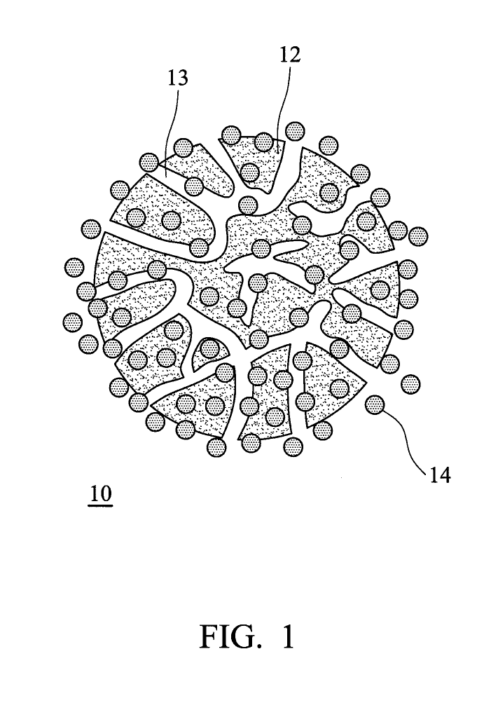

In accordance with an embodiment of the disclosure, a biomedical material is disclosed, as shown in FIG. 1. The biomedical material 10 comprises a biocompatible material 12 and a carrier 14. The carrier 14 is distributed over the surface of the biocompatible material 12. Specifically, both of the biocompatible material 12 and the carrier 14 have no charges, or one of them has charges or both of them have charges with different electricity. The electricity of the carrier 14 can be altered with that of the biocompatible material 12 to ensure the electricity therebetween is different. In an embodiment, the neutral electricity of the carrier can be altered to negative or positive electricity by functionalization. In an embodiment, the carrier 14 and the biocompatible material 12 have a weight ratio of about 1:100,000-1:100, most preferably 1:10,000-1:1,000.

In an embodiment, the biocompatible material 12 may be a porous biocompatible material having a plurality of pores 13. In this embodiment, the carrier 14 may be distributed over the surface or in the pores 13 of the porous biocompatible material 12, as shown in FIG. 1, or encapsulated in the porous biocompatible material 12.

The biocompatible material 12 may comprise metals, metal oxides or metal alloys such as titanium, aluminum, vanadium, cobalt, nickel, chromium, stainless steel or oxides or alloys thereof, polymers such as gelatin, collagen, poly(lactic-co-glycolic acid) (PLGA), poly lactic acid (PLA), poly glycolic acid (PGA), polycaprolactone (PCL), poly methyl methacrylate (PMMA) or elastin, or ceramics such as hydroxyapatite tricalcium phosphate (HATCP), β-tricalcium phosphate (β-TCP), α-tricalcium phosphate (α-TCP), bioactive glass ceramic, calcium sulfate or bone cement.

The biocompatible material 12 may be a non-porous biocompatible material. In this embodiment, the carrier may be distributed on the surface of the non-porous biocompatible material or encapsulated in the non-porous biocompatible material.

The biomedical material 12 may be powder, granulation or scaffold with desired geometry.

The carrier 14 may comprise olein. The olein may comprises phosphatidylcholine (PC) (such as dilinoleoyl phosphatidylcholine (DLPC), dioleoyl phosphatidylcholine (DOPC) or distearoyl phosphatidylcholine (DSPC)), phosphatidylethanolamine (PE) (such as distearoyl phosphatidylethanolamine (DSPE) or dioleoyl phosphatidylethanolamine (DOPE)), 1,2-dioleoyloxy-3-trimethylammonium propane (DOTAP), 2,3-dioleoyloxypropyl-trimethylammonium chloride (DOTMA), phosphatidic acid (PA) (such as dioleoyl phosphatidic acid (DOPA)), phosphatidylserine (PS), phosphatidylglycerol (PG) (such as dioleoyl phosphatidylglycerol (DOPG)), 3β-[N-(N′,N′-dimethylaminoethane)-carbamoyl]cholesterol (DC-CHOL), dihexadecyl phosphate (DHDP) or derivatives thereof. In the carrier 14, the olein has a weight ratio of about 0.1-30 by weight, or 1-15 parts by weight, based on 100 parts by solution weight of the carrier 14. The carrier 14 may further comprise various vitamins such as vitamin A, C, D, E, K, B1, B3, B6, B7 or B12, folate, pantothenic acid or derivatives thereof, or various elements or minerals such as potassium, calcium, iron, magnesium, zinc, copper, manganese, molybdenum, nickel, silicon, chromium, phosphorus, sulfur or chlorine.

The biomedical material 10 may further comprise a bioactive substance (not shown) encapsulated in the carrier 14. The bioactive substance may comprise various growth factors (GF), proteins, peptides, DNA, RNA, cytokines, extracellular matrix (ECM), cell adhesion molecules (CAM), platelets rich plasma (PRP), granulocytes or stem cells. The size of such bioactive substances is from about 2 nm to 10,000 nm.

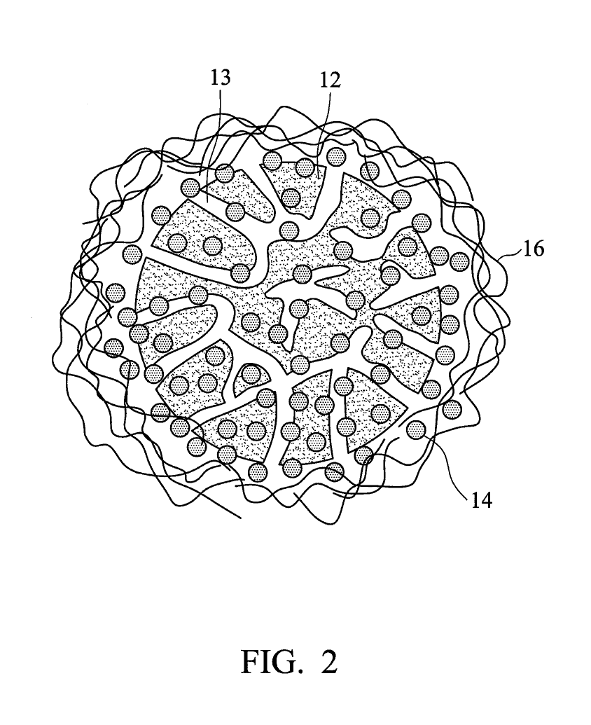

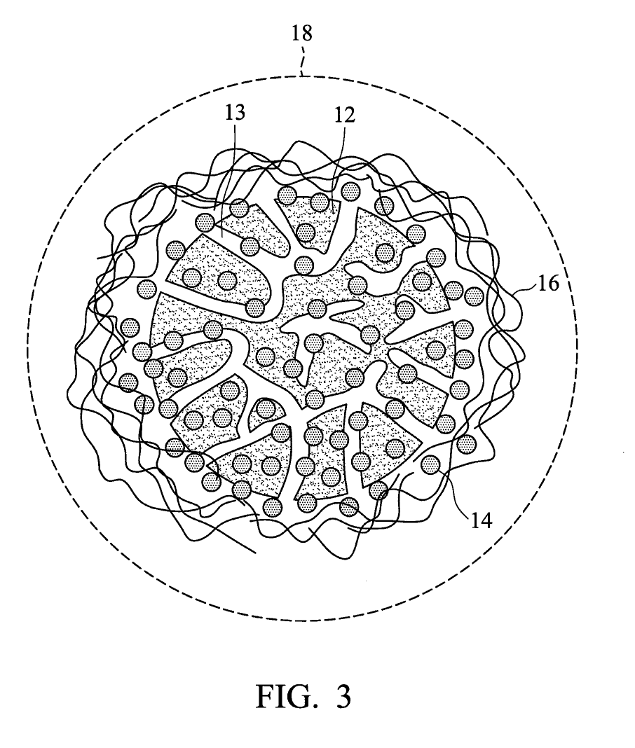

The biomedical material 10 may be further packaged by a polysaccharide layer 16, as shown in FIG. 2. The polysaccharide layer 16 may have positive charges and negative charges, simultaneously. In an embodiment, the polysaccharide layer 16 may comprise, for example, alginate with negative charges and chitosan with positive charges. In an embodiment, the polysaccharide layer 16 may be further packaged by a collagen layer 18, as shown in FIG. 3.

The biomedical material 10 may be a granular structure with a diameter ranging from about 10 to 500 μm. In an embodiment, a plurality of granular structures of the biomedical material may be adhered to one another to form an aggregate by a bio-mount technology using, for example, a biological adhesive. The size of the aggregate may be larger than 50 μm or 1,000 μm.

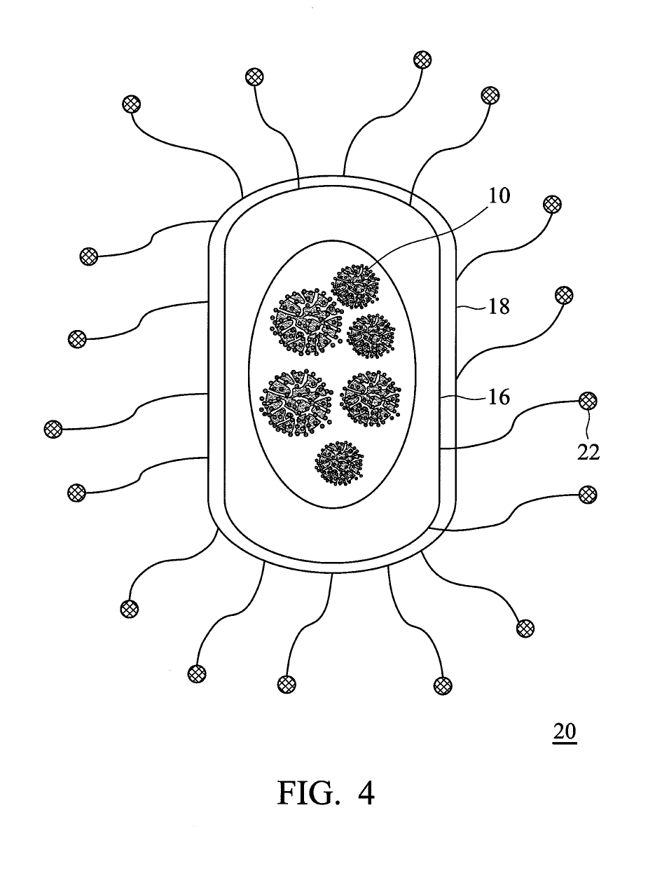

The biomedical material 10, the polysaccharide layer 16 and the collagen layer 18 may be further prepared to form a capsule 20, as shown in FIG. 4, for example, a soft-shell capsule or a hard-shell capsule. In FIG. 4, the number 22 represents an anti-microbial agent.

The microsphere formed by the biomedical material 10, the polysaccharide layer 16 and the collagen layer 18 may be widely utilized for dentistry, orthopedics, wound healing or medical beauty and applied in the repair and regeneration of various soft and hard tissues, for example, applied to various medical fields of dental defects, extraction wounds, combined wounds, small or large bone defects, craniofacial plastic surgery, health beauty or tissue repair.

In accordance with an embodiment of the disclosure, a method for preparing a biomedical material is disclosed. Still referring to FIG. 1, first, a biocompatible material 12 and a carrier 14 are provided. The biocompatible material 12 is then blended with the carrier 14. Specifically, when both of the biocompatible material 12 and the carrier 14 have no charges or one of them has charges, the biocompatible material 12 and the carrier 14 are combined with, for example, a granulation or compaction process. However, when both of the biocompatible material 12 and the carrier 14 have charges with different electricity, the carrier 14 is adsorbed on the surface of the biocompatible material 12 by the different electricity therebetween.

In an embodiment, the biocompatible material 12 may be a porous biocompatible material having a plurality of pores 13. In this embodiment, when both of the porous biocompatible material 12 and the carrier 14 have no charges or one of them has charges, the porous biocompatible material 12 and the carrier 14 are combined with, for example, a granulation or compaction process. However, when both of the porous biocompatible material 12 and the carrier 14 have charges with different electricity, the carrier 14 is adsorbed on the surface or in the pores of the porous biocompatible material 12 by the different electricity therebetween, as shown in FIG. 1. The biocompatible material 12 may comprise metals, metal oxides or metal alloys such as titanium, aluminum, vanadium, cobalt, nickel, chromium, stainless steel or oxides or alloys thereof, polymers such as gelatin, collagen, poly(lactic-co-glycolic acid) (PLGA), poly lactic acid (PLA), poly glycolic acid (PGA), polycaprolactone (PCL), poly methyl methacrylate (PMMA) or elastin, or ceramics such as hydroxyapatite tricalcium phosphate (HATCP), β-tricalcium phosphate (β-TCP), α-tricalcium phosphate (α-TCP), bioactive glass ceramic, calcium sulfate or bone cement.

The carrier 14 may comprise olein. The olein may comprise phosphatidylcholine (PC) (such as dilinoleoyl phosphatidylcholine (DLPC), dioleoyl phosphatidylcholine (DOPC) or distearoyl phosphatidylcholine (DSPC)), phosphatidylethanolamine (PE) (such as distearoyl phosphatidylethanolamine (DSPE) or dioleoyl phosphatidylethanolamine (DOPE)), 1,2-dioleoyloxy-3-trimethylammonium propane (DOTAP), 2,3-dioleoyloxypropyl-trimethylammonium chloride (DOTMA), phosphatidic acid (PA) (such as dioleoyl phosphatidic acid (DOPA)), phosphatidylserine (PS), phosphatidylglycerol (PG) (such as dioleoyl phosphatidylglycerol (DOPG)), 3β[N-(N′,N′-dimethylaminoethane)-carbamoyl]cholesterol (DC-CHOL), dihexadecyl phosphate (DHDP) or derivatives thereof. In the carrier 14, the olein has a weight ratio of about 0.1-30 parts by weight, or 1-15 parts by weight, based on 100 parts by solution weight of the carrier 14. The carrier 14 may further comprise various vitamins such as vitamin A, C, D, E, K, B1, B3, B6, B7 or B 12, folate, pantothenic acid or derivatives thereof, or various elements or minerals such as potassium, calcium, iron, magnesium, zinc, copper, manganese, molybdenum, nickel, silicon, chromium, phosphorus, sulfur or chlorine.

The preparation method may further comprise encapsulating a bioactive substance (not shown) in the carrier 14. The bioactive substance may comprise various growth factors (GF), proteins, peptides, DNA, RNA, cytokines, extracellular matrix (ECM), cell adhesion molecules (CAM), platelets rich plasma (PRP), granulocytes or stem cells.

In an embodiment, when the biomedical material 10 is reserved at −70° C.-26° C., the activity of the bioactive substance can be maintained for at least 35 days.

The biomedical material 10 may be further packaged by a polysaccharide layer 16, as shown in FIG. 2. The polysaccharide layer 16 may have positive charges and negative charges, simultaneously. In an embodiment, the polysaccharide layer 16 may comprise, for example, alginate with negative charges and chitosan with positive charges. In an embodiment, the polysaccharide layer 16 may be further packaged by a collagen layer 18, as shown in FIG. 3.

In accordance with an embodiment of the disclosure, a method for tissue engineering is disclosed. The disclosed biomedical material is provided. The biomedical material is then applied to a targeted tissue for regeneration or healing or a medium for cell growth or cell adhesion. In some embodiments, the biomedical material is applied with a support, for example a membrane or a scaffold, to the targeted tissue or the medium.

One embodiment of the disclosure adopts the nano carries having no charges or having positive/negative charges to be the material encapsulating bioactive substances and to increase the effect and encapsulation rate of the bioactive substances through adjustment of the carrier composition, for instance, by adopting a phosphatidylcholine (PC)/vitamin as the carrier material which can further increase the activity of alkaline phosphatase (ALP) induced by human bone morphogenetic protein 2 (BMP-2). Subsequently, the carriers are adsorbed and fixed on surfaces and in pores of biomedical grade materials (e.g., bioactive glass ceramic or bone cement) through positive/negative charge attraction, or the biocompatible material and the nano carriers, wherein at least one of them has no charges, are combined through granulation or tablet-pressing etc. The biomedical material (e.g., agglomer) of the disclosure can be further coated by a biomedical grade biomedical raw material (ex. polysaccharide or collagen having positive/negative charges simultaneously) (e.g., layer by layer coating) to achieve long-term controlled-release and to effectively protect bioactive substances. The size of the agglomer can be controlled through the size of the above-mentioned biomedical-level material as the core structure (e.g., bioactive glass ceramic or bone cement) while the thickness of the external layer covering the agglomer can be controlled through the number of the assembled layers used and assembly conditions. The microsphere formed by the present biomedical material is easily operated and used in clinical environments, and can be used as a broad-spectrum and instantaneous repair material for bone regeneration, making up for the defects of current products, advantageously evolving into new generation biomedical material products for orthopedics (dental) repair.

In some embodiments of the disclosure, the technology of nano carrier encapsulation combined with the development of the biomedical material (e.g., agglomer) can integrate osteoconduction and osteoinduction and support bone growth, meeting the needs of biomechanics, and further achieving long-term controlled-release, precise quantification of the concentration of bioactive substances and protecting from denaturation of the bioactive substances etc.

In the preparation of the nano carriers, a liposome raw material mainly containing PC is formed into a thin film layer through distillation under reduced pressure, and then encapsulating, for example, PRP, BMP-2 or other bioactive substances, through sonication under low temperature. The charge and interface stability of the carriers are adjusted through different compositions of the liposome in order to be further combined with the biomedical material (e.g., agglomer).

The embodiment of the disclosure adopts the above-mentioned nano carriers and microspheres combined with various biomedical materials to be repair materials for the orthopedics/dental industry. The biomedical materials comprise bioactive glass ceramic, bone cement and collagen etc. Practical applications depend on the purpose of use. The disclosure takes a convenient and simple tablet-pressing technique as an example. However, the practical applications are not limited thereto. Firstly, the powders or particles of various biomedical bone materials (ex. bioactive glass ceramic, HATCP, β-TCP, Ca₂SO₄, gelatin, PLGA etc.) are combined with nano carriers/microspheres encapsulating active factors such as BMP-2, and then bone materials are directly and rapidly prepared through tablet-pressing by a tablet pressing machine. Also, molds with difference shapes can be used to meet each part's needs and the material formulation can be designed pursuant to different needs (ex. adding adhesives, disintegrants, lubricants or anti-disintegrants) to achieve sustained release and improved hardness. The advantages of the tablet-pressing technique comprise: 1. precisely controlling dosages; 2. controlling the drug release rate through the formulation design; 3. ease of mass production and being inexpensive; and 4. being convenient for transportation, preservation and medication.

### EXAMPLE 1

Synthesis of the Vitamin A Derivatives

(1) Preparation of the Vitamin A Ester

First, 100 mg of retinol was dissolved in 2 ml of triethylamine. Fatty acid acyl chloride or fatty acid anhydride with an equal equivalent was then added thereto and stood under room temperature and in the dark. The reactant was analyzed using thin layer chromatography during reaction. After retinol was completely reacted, the reaction solution was poured into water and extracted with ethyl acetate. Ethyl acetate was then separated from the solution. The solution was dehydrated using anhydrous sodium sulfate. After drying and exhausted under reduced pressure, the product was purified using a column.

(2) Preparation of the Alkyl Vitamin A Ester

First, 350 mg of retinoic acid was dissolved in 20 ml of ethyl acetate. Potassium carbonate with an equal equivalent and 2 eq of alkyl iodide were then added thereto with thermal reflux for 2 hours. The reaction solution was cooled, poured into water and washed with water for three times. Ethyl acetate was then separated from the solution. The solution was dehydrated using anhydrous sodium sulfate. After drying and exhausted under reduced pressure, the product was purified using a column.

Vitamin A or vitamin A derivatives were conducted into nano carriers through blending to form ionic bonding or grafting.

### EXAMPLE 2

Preparation of the Nano Carriers (PC/Cholesterol/Vitamin A) Encapsulating Bioactive Substances (BMP-2)

(1) In the example, human bone morphogenetic protein 2 (BMP-2) was encapsulated in nano carriers adopting the thin-film hydration/sonication method. First, a liposome raw material comprising main phosphatidylcholine (PC) and cholesterol, dihexadecyl phosphate (DHDP) and 1,2-dioleoyloxy-3-trimethylammonium propane (DOTAP) was distilled under reduced pressure to form a thin film. BMP-2 was then encapsulated therein through sonication under low temperature (−10° C. to 4° C.). The diameters of the nano carriers encapsulating BMP-2 were smaller than about 200 nm. The weight ratio and composition of olein in the various nano carriers are shown in Table 1. In the example, the charges and interface stability of the nano carriers were adjusted by various nano carrier compositions, facilitating combination with agglomer.

TABLE 1

| | Compositions | | | | |
|---|---|---|---|---|---|
| Formulation | PC | Cholesterol | Charges | | Diameters |
| No. | (weight ratio) | (weight ratio) | (weight ratio) | | (nm) |
| 1 | 10 | 2 | No | | 523 |
| 2 | 10 | 4 | No | | 183 |
| 3 | 10 | 40 | No | | >1,000 |
| 4 | 10 | 20 | +1.5 | (DHDP) | 145 |
| 5 | 10 | 40 | +3 | (DHDP) | >1,000 |
| 6 | 10 | 2 | +1.5 | (DHDP) | 82 |
| 7 | 10 | 2 | +1.5 | (DHDP) | 140 |
| 8 | 10 | 20 | +2 | (DOTAP) | 146 |
| 9 | 10 | 40 | +4 | (DOTAP) | 102 |
| 10 | 10 | 2 | +1.5 | (DHDP) | 124 |
| 11 | 10 | 4 | +4 | (DOTAP) | 92 |

(2) Referring to the weight ratio of formulation No. 11 of Table 1, phosphatidylcholine (PC) was further combined with another olein, for example, phosphatidic acid (PA), phosphatidylethanolamine (PE) or 1,2-dioleoyloxy-3-trimethylammonium propane (DOTAP) and vitamin A to prepare liposomes encapsulating growth factors, as shown in Table 2. The diameters of the nano carriers encapsulating BMP-2 were mainly smaller than about 100 nm.

TABLE 2

| | Compositions (weight ratio) | | | | | | Diameters |
|---|---|---|---|---|---|---|---|
| No. | PC | Cholesterol | PA | DOTAP | PE | Vitamin A | (nm) |
| 1 | 10 | 4 | 10 | | | 0.16 | 55.1 |
| 2 | 10 | 4 | 10 | | | 0 | 48.11 |
| 3 | 10 | 4 | | 5 | | 0.16 | 58.5 |
| 4 | 10 | 4 | | 5 | | 0 | 41.06 |
| 5 | 10 | 4 | | | 5 | 0.16 | 1,303 |
| 6 | 10 | 4 | | | 5 | 0 | 305 |

### EXAMPLE 3

Effect of the Nano Carriers (PC/Cholesterol) Encapsulating Bioactive Substances (BMP-2) on the Activity of an ALP

(1) C₂C₁₂ Cell Culture

C₂C₁₂ cells purchased from Bioresource Collection and Research Center (BCRC) were cultured in an incubator with 5% CO₂ and cryopreserved in a liquid nitrogen barrel. A DMEM medium (10% FBS and 1% penicillin/streptomycin) was utilized for cell culture and subculture. When the cells were cultured to achieve 90% of confluence, the concentration of the cells was diluted with a ratio of 1:10 and subcultured.

(2) Detection of Activity of ALP

The concentration of the C₂C₁₂ cells was adjusted to 4×10⁴ cells/ml. 0.5 ml of the medium was dropped in a 24-well cell culture plate and left to stand in an incubator with 5% CO₂ for 18 hours to make the cells uniformly attach to the cell culture plate. The medium in the cell culture plate attached with the cells was replaced with a DMEM medium (2% FBS) and the sample of No. 4 recited on Table 2 of Example 2 was added thereto. The encapsulation amount of BMP-2 ranged from 10 μg/mL to 100 μg/mL. The cell culture plate was left to stand in the incubator for 72 hours. After the cells were washed with PBS, a lysis buffer was added to the medium. After centrifugation, a supernatant was collected and a BCA (bicinchoninic acid) assay was performed thereon to detect the protein concentration thereof. The activity of alkaline phosphatase (ALP) was detected utilizing the p-nitrophenyl palmitate (pNPP) substrate.

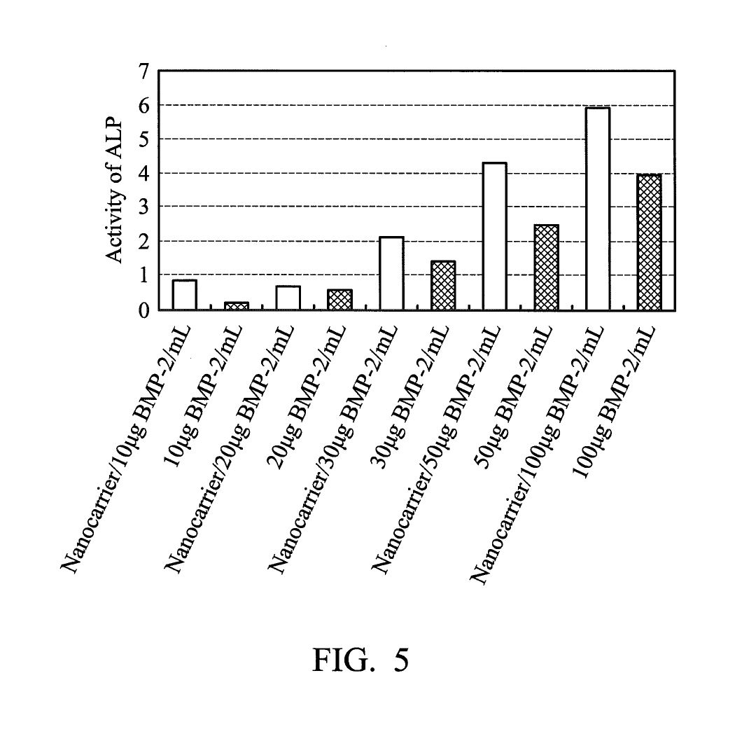

Referring to FIG. 5, the results indicate that the BMP-2 encapsulated by the nano carriers (PC/cholesterol) of the example improved the activity of ALP by about 1.5-1.7 times that of the BMP-2 without encapsulation by nano carriers.

### EXAMPLE 4

Effect of the Nano Carriers (PC/Cholesterol/Vitamin) Encapsulating Bioactive Substances (BMP-2) on the Activity of an ALP

Detection of Activity of ALP

The concentration of the C₂C₁₂ cells was adjusted to 4×10⁴ cells/ml. 0.5 ml of the medium was dropped in a 24-well cell culture plate and left to stand in an incubator with 5% CO₂ for 18 hours to make the cells uniformly attach to the cell culture plate. The medium in the cell culture plate attached with the cells was replaced with a DMEM medium (2% FBS) and the sample of No. 3 recited on Table 2 of Example 2 was added thereto. Other added nano carriers encapsulated various vitamins. The cell culture plate was left to stand in the incubator for 72 hours. After the cells were washed with PBS, a lysis buffer was added to the medium. After centrifugation, a supernatant was collected and a BCA (bicinchoninic acid) assay was performed thereon to detect the protein concentration thereof. The activity of alkaline phosphatase (ALP) was detected utilizing the p-nitrophenyl palmitate (pNPP) substrate.

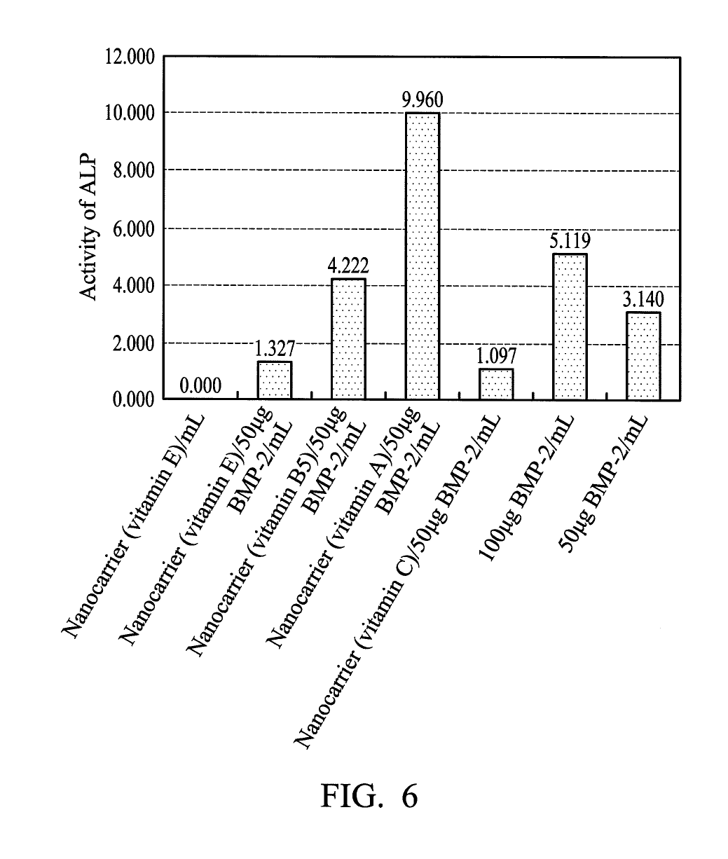

Referring to FIG. 6, the results indicate that the BMP-2 encapsulated by the nano carriers (PC/cholesterol/vitamin A) of the example effectively improved the activity of ALP by about 3 times that of the BMP-2 without encapsulation by nano carriers (from 3.1 to 9.9) and the activity (5.1) of ALP produced by two times that of the amount of the BMP-2 (100 μg/ml) without encapsulation by nano carriers.

### EXAMPLE 5

Effect of the Nano Carriers (PC/Cholesterol/Vitamin A) Encapsulating Bioactive Substances (BMP-2) on the Activity of an ALP

Detection of Activity of ALP

The concentration of the C₂C₁₂ cells was adjusted to 4×10⁴ cells/ml. 0.5 ml of the medium was dropped in a 24-well cell culture plate and left to stand in an incubator with 5% CO₂ for 18 hours to make the cells uniformly attach to the cell culture plate. The medium in the cell culture plate attached with the cells was replaced with a DMEM medium (2% FBS) and the sample of No. 3 recited on Table 2 of Example 2 was added thereto. The sample encapsulated vitamin A with various doses. The cell culture plate was left to stand in the incubator for 72 hours. After the cells were washed with PBS, a lysis buffer was added to the medium. After centrifugation, a supernatant was collected and a BCA (bicinchoninic acid) assay was performed thereon to detect the protein concentration thereof. The activity of alkaline phosphatase (ALP) was detected utilizing the p-nitrophenyl palmitate (pNPP) substrate.

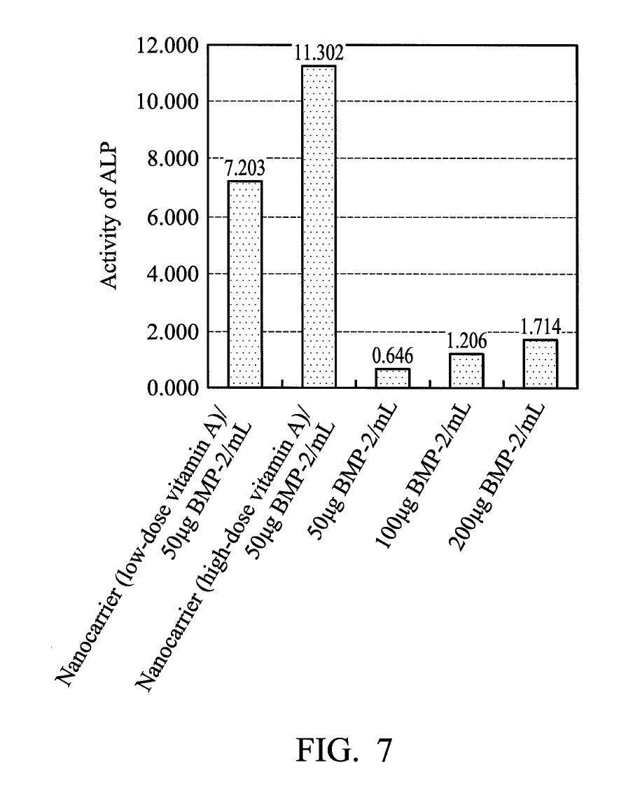

Referring to FIG. 7, the results indicate that the BMP-2 encapsulated by the nano carriers (PC/cholesterol/vitamin A (high dose: 0.26 μμmol/ml)) of the example effectively improved the activity of ALP by about 19 times that of the BMP-2 without encapsulation by nano carriers (from 0.65 to 11.3) and the activity (1.7) of ALP produced by four times that of the amount of the BMP-2 (200 μg/ml) without encapsulation by nano carriers. Therefore, the activity of ALP was apparently increased as the dose of vitamin A of the nano carrier was increased.

### EXAMPLE 6

Alteration of Activity of Bioactive Substances (PRP) Encapsulated by the Nano Carriers (PC/Cholesterol) with Time

Platelets rich plasma (PRP) was respectively encapsulated in the negative-charge nano carriers of No. 2 and the positive-charge nano carriers of No. 4 recited on Table 2 of Example 2 using the method similar to the encapsulation method of BMP-2 and preserved at 4° C. and sampled after 8 days and 35 days, respectively. The contents of TGF-β1 and PDGF-AB were analyzed using an ELISA kit and the content alterations thereof were observed.

PRP, an autologous platelet concentrate, contains various active growth factors, for example, VEGF, PDGF, TGF-β, FGF and etc., which is obtained through separation using a centrifuge, purification and concentration.

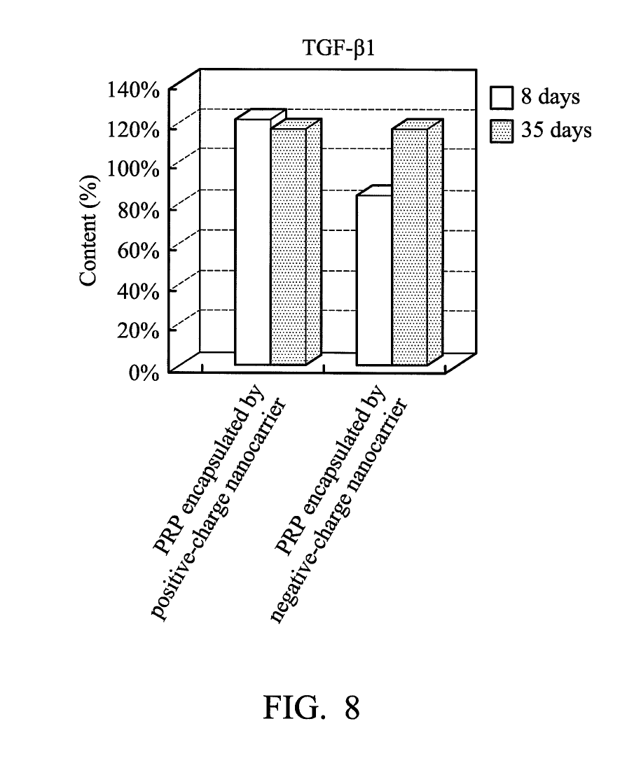

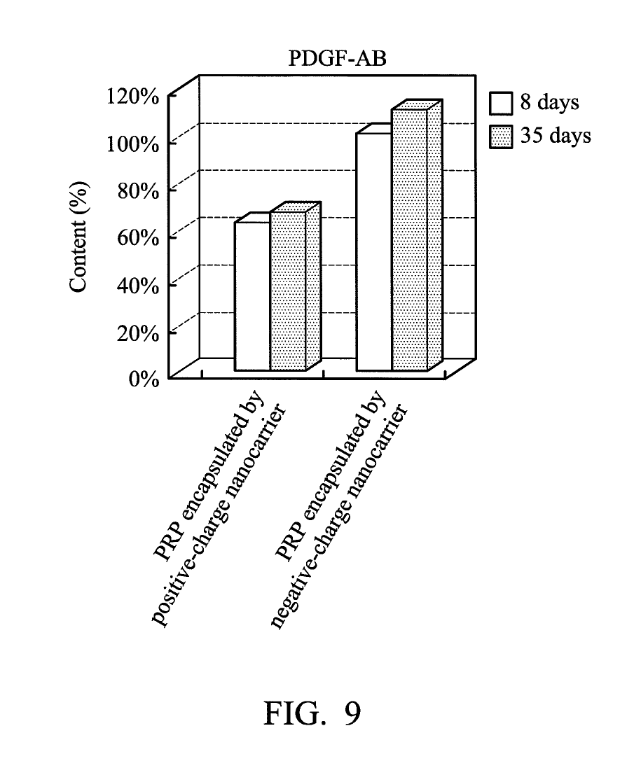

Referring to FIGS. 8 and 9, FIG. 8 shows the alterations of the contents of TGF-β1 of PRP encapsulated by the negative-charge and positive-charge nano carriers with time and FIG. 9 shows the alterations of the contents of PDGF-AB of PRP encapsulated by the negative-charge and positive-charge nano carriers with time. The results indicate that the PRP can be effectively preserved through the nano carriers (PC/cholesterol) of the example. The activity of PRP continued for at least 35 days at 4° C. The shelf life of blood cells in the traditional blood bank is only 7 days and the usual duration of use is about 3 to 5 days before being discarded when expired. In the disclosure, a long-term preservation method for PRP is built.

### EXAMPLE 7

Preparation of the Biomedical Material (Agglomer)

First, a biomedical material such as bioglass, HATCP or calcium sulfate was sieved through various meshes. The biomedical material with 100-600 mesh was selected. The biomedical material with 100-600 mesh was then blended with the nano carriers of No. 1 to 4 recited on Table 2 of Example 2. The ratio of the nano carriers and the biomedical material was 1:20,000.

### EXAMPLE 8

Preparation of the Bioactive Scaffold (Chitosan/Collagen)

First, 1% (w/w) of an acetic acid solution was prepared to form 1-2% of a chitosan solution. The chitosan solution was then blended with collagen solutions with various ratios within a 4° C. reactor to form a blending solution. Next, genipin was added to the blending solution to conduct a cross-linking reaction. The blending solution was then injected into a mold and slowly frozen to −20° C. for 24 hours. Finally, the product was washed with ethanol and water for several times. After freeze-drying, the bioactive scaffold (chitosan/collagen) of the example was obtained.

### EXAMPLE 9

Preparation of the Microsphere

First, 50 mg of porous HATCP was selected as a core structure. 1 ml of the positive-charge nano carriers recited on Table 2 of Example 2 was adsorbed in the porous bioglass as stated for the blending ratio of Example 7. 1-5% of a positive-charge chitosan and 1-5% of a negative-charge alginate were then coated on the biomedical material to form a polysaccharide shell having different charges. Finally, 1-5% of collagen or gelatin was coated thereon to prepare the multi-layered microsphere structure of the example.

### EXAMPLE 10

Controlled Release of the Biomedical Material (Agglomer)

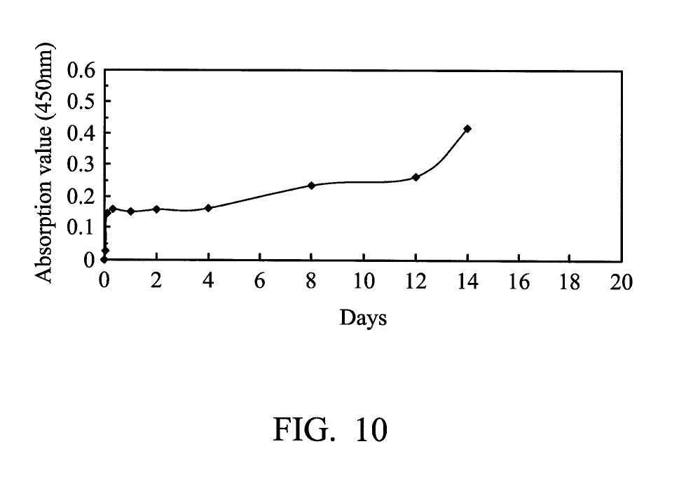

The agglomer prepared by Example 7 was placed in water-soluble buffer aqueous buffer solution and then the controlled release condition thereof was observed and respectively sampled at the starting point, 1^{st} hour, 3^{rd} hour, 8^{th} hour, 1^{st} day, 2^{nd} day, 4^{th} day, 8^{th} day and 14^{th} day. After sampling, the samples were analyzed using an ELISA, and the results are shown in FIG. 10.

The results indicate that when the agglomer was placed in the aqueous solution at the initial stage, a portion of the BMP-2 was released. A burst release of BMP-2 was observed. After 4 days, BMP-2 was more significantly released. After 12 days, BMP-2 was released in a great quantity. That is, the release of BMP-2 was controlled by the agglomer for more than 14 days. When OD₄₅₀ (theoretical value) was 1.48, the release rate was 100%.

### EXAMPLE 11

Effect of the Biomedical Material (Agglomer) on the Activity of an ALP

The concentration of the C₂C₁₂ cells was adjusted to 4×10⁴ cells/ml. 1.0 ml of the medium was dropped in a 12-well cell culture plate and left to stand in an incubator with 5% CO₂ for 18 hours to make the cells uniformly attach to the cell culture plate. The medium in the cell culture plate attached with the cells was replaced with a DMEM medium (2% FBS). A transwell with a pore size of 8.0 μm was placed on the cell culture plate. The biomedical material (agglomer) of Example 7 was placed in the transwell. The medium was then added to the transwell to cover the biomedical material (agglomer). The cell culture plate was left to stand in the incubator for 72 hours. After the cells were washed with PBS, a lysis buffer was added to the medium. After centrifugation, a supernatant was collected and a BCA (bicinchoninic acid) assay was performed thereon to detect the protein concentration thereof. The activity of alkaline phosphatase (ALP) was detected utilizing the p-nitrophenyl palmitate (pNPP) substrate.

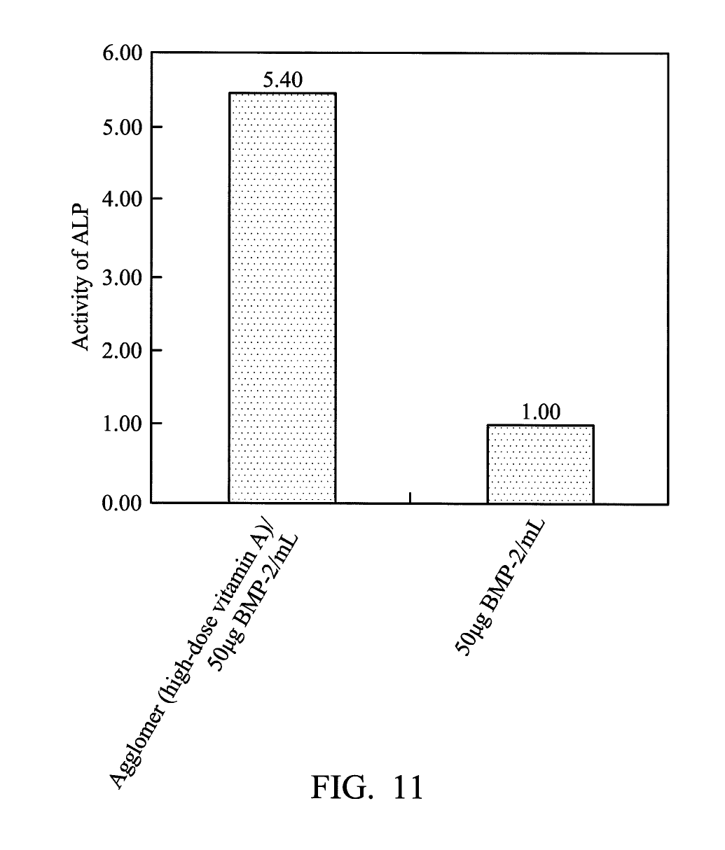

Referring to FIG. 11, the results indicate that the BMP-2 encapsulated by the nano carriers (PC/cholesterol/vitamin A (high dose: 0.26 μμmol/ml)) prepared by Example 5 of the agglomer improved the activity of ALP by about 5 times that of the free BMP-2 (from 1.00 to 5.40).

### EXAMPLE 12

Effect of the Biomedical Material (Agglomer) on Bone Repair

The positive-charge nano carriers prepared by Example 2 were blended with a negative-charge bioglass (200 μm) to form an agglomer. The agglomer (similar to Example 7, but the ratio of the nano carriers and the biomedical material was adjusted to 1:7,000) was used to perform an animal experiment. A bone with a size of 5 mm*5 mm was cut from a rat by an implement and observed for 12 weeks. The test materials comprising the first generation repair materials were used to verify the effect of the nano carriers of the disclosure. In the example, the conditions of bone repair were compared between the nano carriers combined with negative-charge biomedical materials approved by the FDA and other control groups.

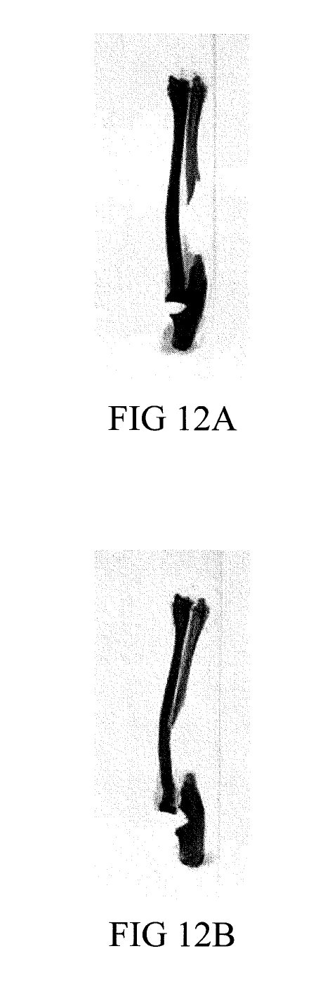

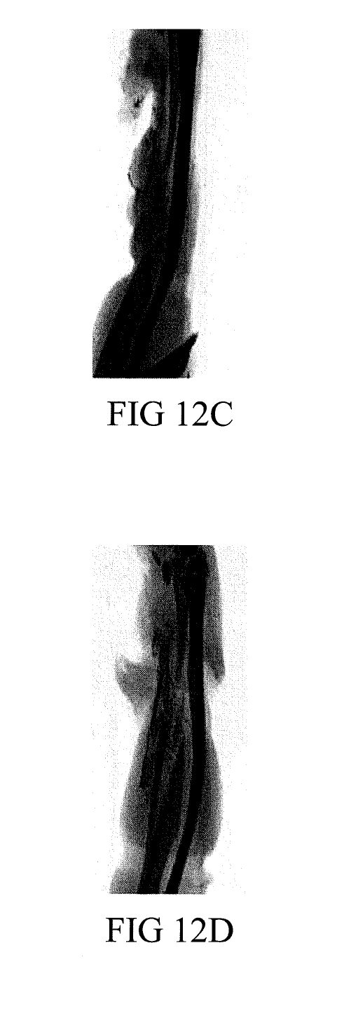

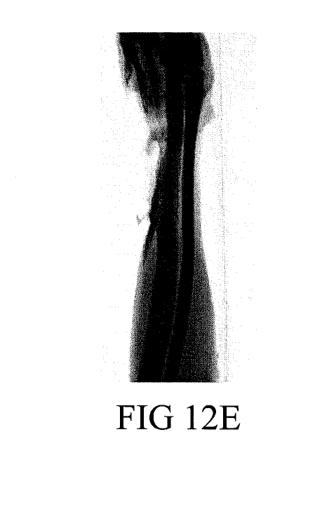

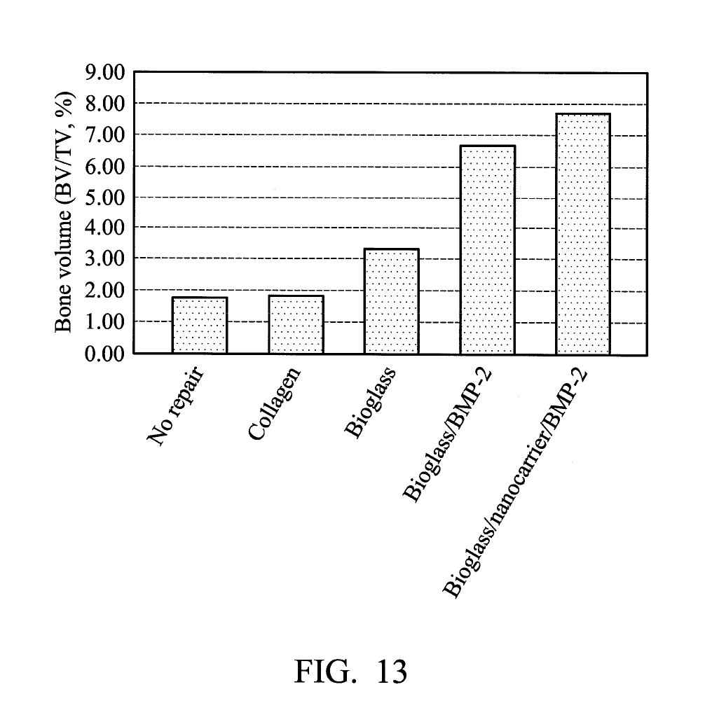

After 12 weeks, the condition of bone repair was detected using an x-ray. The results indicated that, in the control group wherein only collagen was used, no bone repair was presented (as shown in FIG. 12B). In another control group wherein only bioglass was used, slight bone repair was presented (as shown in FIG. 12C). However, in the group wherein the nano carries were added, apparent bone repair was presented (as shown in FIG. 12E); especially, at the position of the gap junction, wherein the bone repair condition thereof was better than that of the group wherein non-encapsulated BMP-2/bioglass was used (as shown in FIG. 12D). Therefore, using the assembly of bioglass/nano carries, a better bone repair condition was achieved. However, the density of bone repair thereof was similar to that of the group wherein non-encapsulated BMP-2/bioglass was used. Therefore, the assembly of bioglass/nano carries can be replaced with agglomer/nano carriers (PC/vitamin/BMP-2). The density of bone repair and osseointegration were improved due to the increased effects of BMP-2 and sustained release produced by the agglomer, and the amount of usage of BMP-2 was simultaneously reduced. In accordance with the micro-CT image analysis, the results regarding healing and repair of bone defect within 12 weeks are shown in FIG. 13. The groups which used the BMP-2 had the largest increase in bone volume (=BV/TV); particularly, for the group which used the nano carriers encapsulating growth factors (BMP-2).

### EXAMPLE 13

Properties of the Biomedical Material (Agglomer) Tablet

To successfully combine the nano carriers and microsphere with various biomedical materials and achieve sustained release or increase hardness through addition of various excipients in accordance with the requirements, in the example, the nano carriers, various biomedical bone materials (the main material) (bioglass, HATCP and β-TCP) and excipient (ex. adhesive (such as cellulose, carboxymethyl cellulose, methyl cellulose, sodium alginate or gelatin) and lubricant (such as magnesium stearate and silicon dioxide)) were uniformly blended with various ratios (compositions and ratios of various formulations were shown in Table 3) and compressed into tablets. The biomedical materials utilized by the disclosure are not limited to porous biomedical materials, for example, the main material of non-porous β-TCP as shown in Table 6. The following results indicate that the nano carriers can be combined with the biomedical materials and encapsulated thereby. The weight and hardness of the tablets, after compressing, are shown in Table 4 to Table 6.

TABLE 3

| | Main | Cellulose | Magnesium stearate | Silicon dioxide |
|---|---|---|---|---|
| | material | (adhesive) | (lubricant I) | (lubricant II) |
| Formulation I | 85% | 5% | 5% | 5% |
| Formulation II | 70% | 20% | 5% | 5% |
| Formulation III | 40% | 50% | 5% | 5% |

*The main material contains 5% of nano carriers.

TABLE 4
(the main material of bioglass)

| | Formulation I | Formulation II | Formulation III |
|---|---|---|---|
| Weight (mg) | 581.1 | 490.8 | 329.9 |
| Hardness (kg) | 1.56 | 2.08 | 2.24 |

TABLE 5
(the main material of HATCP)

| | Formulation I | Formulation II | Formulation III |
|---|---|---|---|
| Weight (mg) | 558.1 | 399.5 | 342.0 |
| Hardness (kg) | 1.32 | 1.21 | 4.55 |

TABLE 6
(the main material of non-porous β-TCP)

| | Formulation I | Formulation II | Formulation III |
|---|---|---|---|
| Weight (mg) | 731.3 | 443.6 | 407.6 |
| Hardness (kg) | 4.30 | 1.73 | 5.84 |

The test results indicate that the nano carriers and microsphere can be successfully combined with various biomedical materials and the hardness of the tablets can be controlled through adjustment of the formulations in accordance with requirements. If necessary, disintegrants can also be added to control the drug release rate. Additionally, the surface modification technique, for example, the film-coating method can be utilized or added other materials to achieve the effect of protection and sustained release.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for tissue engineering, comprising:
providing a biomedical material comprising a biocompatible material having a surface and a carrier distributed over the surface of the biocompatible material, wherein both of the biocompatible material and the carrier have no charges, one of them has charges or both of them have different electrical charges, wherein the carrier and the biocompatible material have a weight ratio of 1:100,000-1:100; and
applying the biomedical material to a targeted tissue for regeneration or healing or a medium for cell growth or cell adhesion.

2. The method as claimed in claim 1, wherein the carrier and the biocompatible material have a weight ratio of 1:10,000-1:1,000.

3. The method as claimed in claim 1, wherein the biocompatible material is in the form of powder, granulation or scaffold.

4. The method as claimed in claim 1, wherein the biocompatible material is a porous or non-porous biocompatible material.

5. The method as claimed in claim 4, wherein the carrier is further distributed in the pores of the porous biocompatible material or encapsulated in the porous biocompatible material.

6. The method as claimed in claim 4, wherein the carrier is further distributed on the surface of the non-porous biocompatible material or encapsulated in the non-porous biocompatible material.

7. The method as claimed in claim 1, wherein the biocompatible material comprises metals, metal oxides, metal alloys, polymers or ceramics.

8. The method as claimed in claim 7, wherein the metals, metal oxides or metal alloys comprise titanium, aluminum, vanadium, cobalt, nickel, chromium, stainless steel or oxides or alloys thereof.

9. The method as claimed in claim 7, wherein the ceramics comprise hydroxyapatite tricalcium phosphate (HATCP), β-tricalcium phosphate (β-TCP), α-tricalcium phosphate (α-TCP), bioactive glass ceramic, calcium sulfate or bone cement.

10. The method as claimed in claim 7, wherein the polymers comprise gelatin, collagen, poly(lactic-co-glycolic acid) (PLGA), poly lactic acid (PLA), poly glycolic acid (PGA), polycaprolactone (PCL), poly methyl methacrylate (PMMA) or elastin.

11. The method as claimed in claim 1, wherein the carrier comprises olein.

12. The method as claimed in claim 11, wherein the olein comprises phosphatidylcholine (PC), phosphatidylethanolamine (PE), 1,2-dioleoyloxy-3-trimethylammonium propane (DOTAP), 2,3-dioleoyloxypropyl-trimethylammonium chloride (DOTMA), phosphatidic acid (PA), phosphatidylserine (PS), phosphatidylglycerol (PG),3β-[N-(N′,N′-dimethylaminoethane)-carbamoyl]cholesterol (DC-CHOL), dihexadecyl phosphate (DHDP) or derivatives thereof.

13. The method as claimed in claim 11, wherein the olein has a weight ratio of 0.1-30 parts by weight, based on 100 parts by solution weight of the carrier.

14. The method as claimed in claim 11, wherein the carrier further comprises vitamin A, C, D, E, K, B1, B3, B6, B7, B12, folate, pantothenic acid or derivatives thereof.

15. The method as claimed in claim 11, wherein the carrier further comprises potassium, calcium, iron, magnesium, zinc, copper, manganese, molybdenum, nickel, silicon, chromium, phosphorus, sulfur or chlorine.

16. The method as claimed in claim 1, further comprising encapsulating a bioactive substance in the carrier.

17. The method as claimed in claim 16, wherein the bioactive substance comprises growth factors, proteins, peptides, DNA or RNA.

18. The method as claimed in claim 16, wherein the bioactive substance comprises cytokines, extracellular matrix (ECM) or cell adhesion molecules (CAM).

19. The method as claimed in claim 16, wherein the bioactive substance comprises platelets rich plasma (PRP), granulocytes or stem cells.

20. The method as claimed in claim 1, further comprising coating a polysaccharide layer on the biomedical material.

21. The method as claimed in claim 20, wherein the polysaccharide layer has positive charges and negative charges.

22. The method for tissue engineering as claimed in claim 1, further comprising applying the biomedical material with a support to the targeted tissue or the medium.

23. The method for tissue engineering as claimed in claim 22, wherein the support comprises a membrane or a scaffold.
